(19) United States
(12) Patent Application Publication
(10) Pub. No.: US 2009/0308399 A1
(43) Pub. Date: Dec. 17, 2009

(54) STD BARRIER

(75) Inventors: Carol A. NIEMI, Houston, TX (US); Elaine K. MAYS, Houston, TX (US)

Correspondence Address: BAKER & MCKENZIE LLP, Pennzoil Place, South Tower, 711 Louisiana, Suite 3400, HOUSTON, TX, 77002-2716, US

(73) Assignee: Carelaine LLC, Houston, TX (US)

(21) Appl. No.: 12/483,464

(22) Filed: Jun. 12, 2009

Related U.S. Application Data
(60) Provisional application No. 61/061,177, filed on Jun. 13, 2008.
(60) Provisional application No. 61/143,209, filed on Jan. 8, 2009.

(51) Int. Cl. A61F 13/00

(52) U.S. Cl. 128/830

(57) ABSTRACT

The invention generally relates to an sexually transmitted disease (STD) barrier, comprising a planar sheet of biocompatible material having a durometer of less than 85, a tensile strength of at least 3000 psi, a reversible elongation stretch of at least 150%, and that excludes virus and microbes when tested at body temperature and under load.

### PRIOR RELATED APPLICATIONS

This application claim priority to U.S. Provisional Application 61/061,177, filed Jun. 13, 2008, and 61/143,209 filed Jan. 8, 2009, each expressly incorporated by reference.

### FEDERALLY SPONSORED RESEARCH STATEMENT

Not applicable.

### REFERENCE TO MICROFICHE APPENDIX

Not applicable.

### FIELD OF THE INVENTION

The invention relates to the prevention of disease transmission during intimate sexual activity.

### BACKGROUND OF THE INVENTION

There has been considerable study of prevention of disease and pregnancy, and a wide variety of rubber and natural prophylactics (condoms) for such use are available at reasonable cost in the Western world. However, these devices are not suitable for a variety of sexual activities that do not involve intercourse, in particular oral sexual activities. Yet, even these activities can lead to disease transmission, and an affordable device that prevents sexually transmitted diseases (STDs) would be of great benefit.

Current methods of preventing disease transmission during oral sexual activities in common use include latex dental dams, self-modified exam gloves and off-label use of various food wraps. However, such devices are less than optimal, being inconvenient to use or having a bad taste or sensation transmission characteristics. Further, the latex devices taste very bitter and many people have latex allergies, preventing their wide-spread adoption.

Further, although Project Start reports that 12% of incarcerated men in the United States are using SARAN WRAP™ and other plastic food wraps as a means of protection during consensual sex, and an article in the Journal of the Gay and Lesbian Medical Association reports that 2% of lesbian and bisexual women report using plastic wrap for oral sex, there is NO evidence that such films stop the transmission of diseases, particularly when used under load. Further, the extensive off-label use of plastic wrap such as SARAN WRAP™ for disease prevention suggest that a proven and inexpensive STD barrier is much needed in the art.

There have been patents directed to various efforts to solve the difficulty of preventing disease transmission during oral sex. For example, U.S. Pat. No. 4,949,731 discloses an oral prophylactic device comprising elastic flexible material to conform to the mouth. U.S. Pat. No. 5,409,016 discloses another prophylactic device for oral sex that also conforms to mouth, and can be made of latex or polyurethane material. This device has fasteners that hook behind the ears to keep the barrier in place during use. Several other patents are directed to similar devices, including U.S. Pat. No. 5,655,543, U.S. Pat. No. 5,513,653, JP8141008, U.S. Pat. No. 6,418,931 and GR1002601. However, all of these devices are of complex topology making them expensive to manufacture and therefore less practical for wide spread usage. They also render the user less visually appealing, an important consideration for intimate purposes, which depends on consistent and proper use.

There have been a few attempts to design a simpler STD barrier for oral use. U.S. Pat. No. 5,649,549, for example, describes a contraceptive device for oral sex including a body portion having a rounded upper portion, a tapered lower portion, and an intermediate portion therebetween. A plurality of nubs extend outwardly from the intermediate portion of the body portion, and supposedly secure over the genital area with the plurality of nubs disposed over the clitoral area to allow for safe oral stimulation thereof.

U.S. Pat. No. 4,735,621 describes a female “condom” having a thin-walled, condom-like tubular protective device.

CN1230392 describes a contraceptive article in the shape of underwear that is made of polyurethane material and/or high grade emulsion, that is water tight and air tight in the part corresponding to reproductive organs. Such an article would be aesthetically unappealing (reminiscent of adult diapers) and likely to cause sweating.

JP7178129 describes STD barriers formed by sticking together a first and second filmy, detachable sheets, wherein the first filmy sheet is used for encircling the penis and the second filmy sheet is used for shielding the periphery of the vagina or being fitted in the vagina.

U.S. Pat. No. 5,534,346 describes a disposable, general purpose barrier to aid in preventing cross-contamination from one surgical operation to another. This barrier is visually transparent or translucent, flexible, pliable, rectangular, thin-film provided with a coating of a low-tack pressure sensitive adhesive over a select area for attachment to the patient's skin. However, this device would probably not be suitable for intimate use, not being optimized for the transmission of sensation, or having objectionable taste. Furthermore, there is no testing of these devices for viral penetration, and thus their efficacy for STD prevention is in doubt.

A sheet barrier is available under the name HOT DAM.™ It is advertised as a non-latex sheet that is an “FDA approved” barrier. However, a search of the FDA website indicates that the sheets are not approved, and the manufacturer has confirmed that it is not approved. Thus, there is no information indicating that these devices provide an effective viral barrier.

Therefore, what is needed in the art is an STD barrier that is inexpensive to manufacture, and has optimal taste, flexibility and durability for use in intimate activities. Ideally, the device could be manufactured and sold for small cost, and be packaged in a way to as to maximize convenience.

### SUMMARY OF THE INVENTION

The invention relates generally to a highly portable, easy to dispense and attractively packaged personal protective device, known as “PERSONAL PLASTIC™,” that guards against the transmission of various diseases for use in a range of sexual activities. The product can use existing formulations of polymer film, preferably polyurethane, keratin, nitrile, neoprene, vinyl or other plastic films of similar properties, packaged for ease of personal use. The intent is disease prevention via a material that allows more tactile responsiveness than currently available latex dental dams, better taste than latex, and better guarantees of protection than off-label uses of food wraps, presented in an attractive and discreet dispenser.

More particularly, the invention is a sexually transmitted disease (STD) barrier, comprising a planar sheet of biocompatible material having essentially no taste, a durometer of less than 85, a tensile strength of at least 3000 psi, a reversible elongation stretch of at least 150%, that excludes virus and microbes when tested at 37° C., and preferably protects against viral penetration at 150% stretch and higher loads.

In a preferred embodiment, the STD barrier is a 12×12 sheet of biocompatible polyurethane film, having about 20-200 um thickness, essentially no taste, a durometer of less than 85 and preferably about 70, a tensile strength of at least 3000 psi, a reversible elongation stretch of at least 150%, and that excludes virus and microbes when tested at 37° C. and also when stretched to 150% or more. In another preferred embodiment, the STD barrier is a keratin or keratin-silicone copolymer film with similar properties.

### BRIEF DESCRIPTION OF THE DRAWINGS

FIG. 1. Shows a top view of a square and hourglass STD barriers.

FIG. 2. Shows a side view of interleaved STD barriers.

FIG. 3 shows STD barriers having tabs for easy dispensing of the barriers.

FIG. 4 shows one or more dispensers.

FIG. 5 shows the experimental design.

FIG. 6 shows a schematic of the samples tested in the viral barrier experiment.

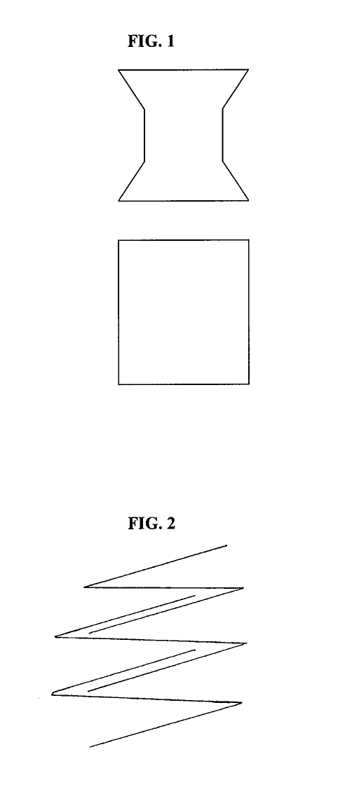

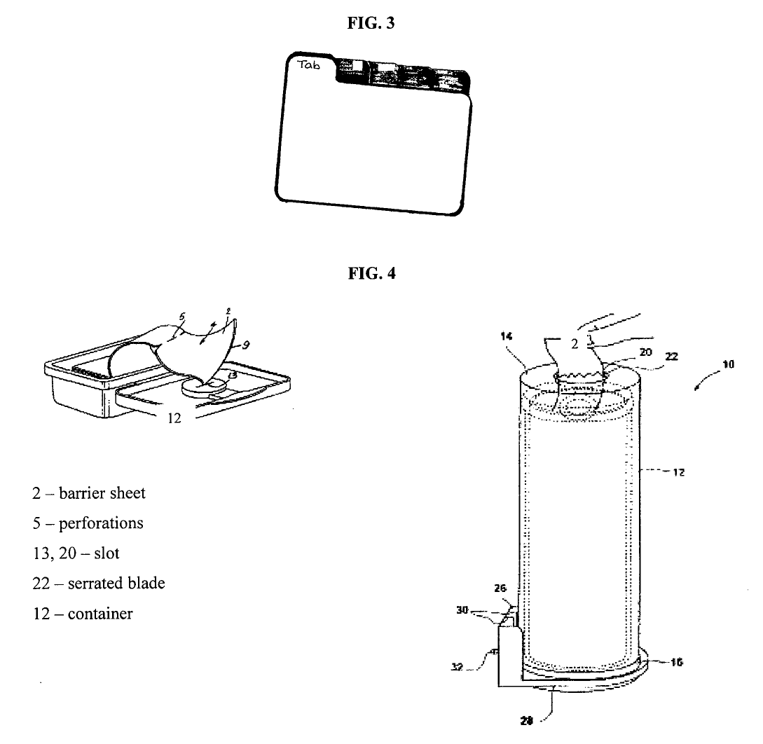

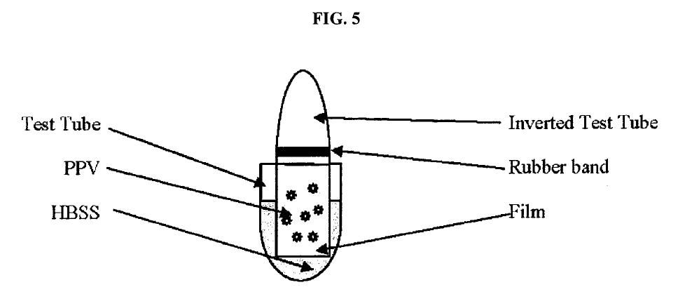

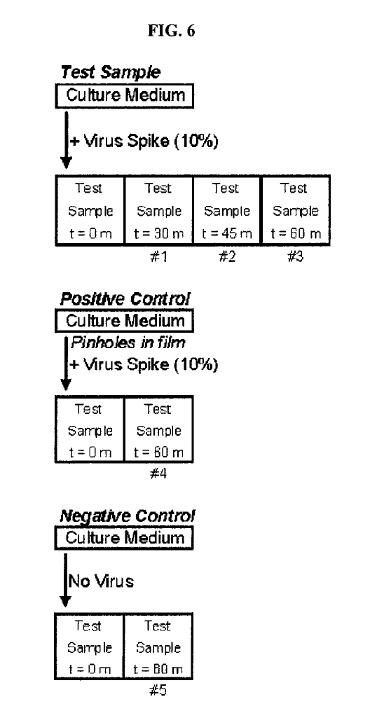

### DESCRIPTION OF EMBODIMENTS OF THE INVENTION

The invention provides a novel method of preventing disease transmission during oral and other sexual acts using a thin STD barrier, that is distinguished from latex “dental dams” and off-label use of polyethylene film food wrap. Preferably the STD barrier is made of keratin plastic film, which makes use of waste material, is sustainable and ultimately biodegradable, or nitrile film, which offers better chemical resistance, or neoprene or polyurethane film, which is more economical. All of these materials are more aesthetically pleasing than latex film (which is bitter and allergenic) and provide similar or superior barrier, strength and durability and puncture resistance properties. Preferably, the STD barrier is precut to an optimal size and dispensed in a durable container for portability and ease of use.

In a preferred embodiment, the STD barrier will be dispensed in scored or precut pieces approximately 6-16 inches square, preferably about 12 inches square, in a durable and attractive box with no externally visible label. Product name and instructions for use will be printed on the inside cover of the box. The box can be encased in a plastic film sleeve printed with product name and general usage information for ease of sale and shelf durability, but the sleeve can be removed after purchase for maximum discretion.

This product is intended to allow more sensation and be more aesthetically pleasing than latex dental dams, and more reliably effective than off-label use of polyethylene film (Saran Wrap and similar products). Like condoms, this product will be available as widely as possible, at convenience stores, drug stores, grocery stores and adult stores, as well as online.

Polyurethanes are among the most versatile construction materials that can be formulated for medical devices. They are tough, biocompatible, and hemocompatible. They can be strong elastomers or rigid plastics, and they can be processed using extrusion, injection molding, film blowing, solution dipping, and two-part liquid molding. It is their unique chemistry gives polyurethanes this versatility. They are segmented polymers, meaning they have a soft segment that provides flexibility and a hard segment that provides strength. Polyurethanes are made from three basic building blocks: the backbone, the diisocyanate, and the chain extender. The backbone, usually a long chain molecule, provides flexibility to the polymer. The diisocyanate and the chain extender combine to form the hard segment, which acts as a cross-link. It provides the polymer with high tensile strength and high elongation.

Thermoplastic polyurethanes (TPU) contain no plasticizer, yet offer toughness and strength, and high water-vapor permeability. TPUs are thermoplastic rubbers made from isocyanates and are designated aromatic or aliphatic on the basis of the chemical nature of the diisocyanate component in their formulation. Aromatic and aliphatic polyurethanes share the following properties that make them suitable for use in medical devices:

High tensile strength (3000 psi).
High ultimate elongation (250-700%).
Wide range of durometer (70 Shore A to 84 Shore D).
Good biocompatibility.
High abrasion resistance.
Good hydrolytic stability.
Ability to be sterilized via ethanol, gamma or uv irradiation.
Ability to retain elastomeric properties at low temperature.
Resistance to chemicals, oil, and UV light.
Several TPU polymers are already FDA approved or cleared for use in food and medical devices.

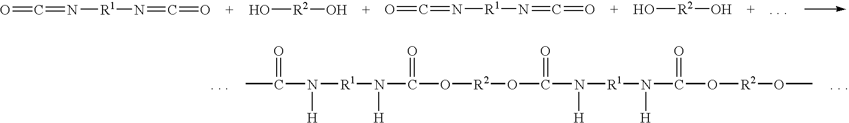

Keratins are a family of structural proteins found in hair and nails that provide support and form in all eukaryotic biologic systems. Originally thought to be antigenic, keratin is now known to be completely biocompatible, as evidenced by the discovery of a lock of hair that had been buried in the soft tissues of a plastic surgery patient for ten years without scar tissue or inflammation of any sort. Keratin is the ultimate renewable resource, available for free at any military installation. Further, it can be harvested and formed into thin sheets, such as are required for the instant application.

Patents describing the harvesting of keratin and the formation of biocompatible film products include at least the following: U.S. Pat. No. 5,358,935, U.S. Pat. No. 5,948,432, U.S. Pat. No. 6,124,265, U.S. Pat. No. 6,914,126, U.S. Pat. No. 6,989,437, and related patents. Further, keratin based products can be combined with other copolymers as in US200482717 (describing keratin-silicone copolymers). Keratin films are available at KERAPLAST TECHNOLOGIES™ LTD.

In a preferred embodiment, the material of the invention is a thermoplastic polyurethane or keratin film, but another biocompatible film such as nitrile or neoprene can be used provided it has the following characteristics:

Durometer: The durometer of the STD barrier is 65-85, and preferably 70-80 or about 75. Durometer is the measure of the hardness of a particular material as measured on the Shore A scale (the A scale is for softer plastics) under ASTM D2240 or ISO 868. The higher durometer grades yield a very tough product with very high puncture resistance and very high abrasion resistance. The lower durometer grades, such as 70-75, exhibit a very supple quality and feel very soft against human skin, making it an ideal for intimate uses.

Elongation: The STD barrier of the invention should have a very high elasticity, and allow at least 150% stretch before breaking. However, with polyurethane, it is possible to achieve 200%, 300%, 400%, 500%, and even up to 800% elongation before breaking. In preferred embodiments, the material will also have excellent memory, meaning that it will return to near original dimensions after being stretched to its limit.

Tensile Strength: The STD barrier of the invention must have high tensile strength of at least 3000 psi. In preferred embodiment the strength can be even higher, ranging from 4000, 5000, 6000, 7000, 8000 and up to 9000 psi. Tensile properties can be measured under ISO 527-3 or ASTM D882-02.

Long-Term Durability: Polyurethane is manufactured without the use of plasticizers, which means it will retain its original performance characteristics for longer time periods. Not having plasticizers also means it will not leach out hazardous compounds, which is of particular importance for intimate use.

Biocompatibility: It is important that the film of the invention be biocompatible.

Anti-Microbial/Anti-viral: In some embodiments, the STD barrier may include antibacterial or antiviral additives, provided that the additive does not negatively effect taste, or biocompatibility. For example, zinc-containing lubricants can be coated onto once side of the STD barrier during manufacture.

Viral and Microbial Penetration: It is critical to the invention that the STD barrier not allow the penetration of viruses both with and without load. Viral penetration is measured using the porcine parvovirus (PPV). This virus has a diameter of 18-24 nm and is similar in size to the smallest of human pathogens.

Chemical Resistance: In preferred embodiments, the STD barrier is resistant to degradation by common lubricants, gels, flavorings and the like.

Thickness: The thinner the barrier, the better the tactile responsiveness. However, this quality must be balanced against strength, durability and viral and microbial exclusion. Thus, a variety of thickness are possible depending on the characteristics of a given film. However, generally a thickness of about 20-200 um will suffice, although other thicknesses may also function well in the invention. Thicknesses range from 0.0001, 0.0005, 0.001, 0.005, 0.01, 0.02, 0.05, to 0.1 mm or more, depending on the material and viral penetration.

Possible films for use in the invention therefore include low density polyethylene (LDPE), polyvinylidene chloride (PVDC), linear low density polyethylene (LLDPE), polyisobutene (PIB), and poly[ethylene-vinylacetate] (EVA) copolymers, nitrile, neoprene, vinyl, and the like, provided that they are optimized to have the claimed requirements. The preferred film is a polyurethane film, in particular aromatic polyether polyurethane films, or a keratin or keratin copolymer film.

Packaging is very important to the commercial success of an STD barrier. As mentioned above, the packaging should be discrete, but it should also a dispense single sheet without contaminating the rest of the sheets by hand contact, and the sheet should be dispensed without self-tangling or self-clinging.

In a simple embodiment, the sheets are folded and simply stacked one on top of the next in a hinged box. In another the sheets are folded and interleaved along a small portion so that removal of a single sheet pulls the edge of the next sheet out of the dispenser in the same way that Kleenex is dispensed. The optimal degree of overlap vary with the dinginess of the plastic and can easily be determined empirically.

U.S. Pat. No. 6,053,357 describes a “pop-up” box dispenser having a curvilinear opening for dispensing thin sheets of tissue. Typically, the curvilinear opening is incorporated into a thin plastic film overlaid on the top of a cardboard box. WO0065972 describes a similar box dispenser with central opening.

US2004169047 describes a dispenser for inter-folded sheet including a replaceable cartridge. In preferred embodiments, the replaceable cartridge includes a generally planar rectangular core having a stack of inter-folded food service sheets wrapped around the core in a U-shape and retained in position by shrink wrap. The replaceable cartridge can be sold in a plastic film wrapping, further reducing the cost of the device.

In another embodiment, a simple roll dispenser is used with perforated roll of film. There are many suitable designs available for roll dispensers, including U.S. Pat. No. D452,788S, U.S. Pat. No. 5,768,968, and U.S. Pat. No. 4,340,162, and the dispenser can be with or without cutting edges as needed for easy dispensing.

U.S. Pat. No. 7,216,775 describes a napkin dispenser whereby the napkins are dispensed through a central opening in a jar shaped container, whereby varying the orifice diameter, co-molded density and stiffness or geometry of the actual lobes defining the dispensing aperture allows the precise amount of friction to be created in the dispensing opening for selectively grabbing or releasing the towelette, thereby tearing the towelette connecting perforations at just the right time. WO2004052164 describes a similar device.

JP2007244588 describes a cosmetic oil-blotting sheet package. It is a small cardboard envelope-style folder with a slot and tab for secure closure. Inside the flap of the envelope is a small area of double-sided tape. When a sheet of film is desired, the flap is pressed in the correct area and tape sticks to a piece of film pulling it out of the dispenser.

U.S. Pat. No. 5,664,677 describes dispenser containers with a sealable flap top. The assembly comprises a generally flat container having therein a plurality of wipes therein. A top portion of the container defines an opening for wiper cloth removal which may be selectively opened and closed using a flexible adhesive panel. The bottom portion of the container is characterized by a greater rigidity than the rigidity of the top portion. This enhanced rigidity is sufficient to substantially prevent bending of the container during removal of the wiper cloths and closure of the container opening. This device could also be modified to include a larger opening, and double-sided tape added to flap top, such that pressing the flap into the container, pulls up a single sheet of film therethrough.

Another dispenser is described in US2006060599 and includes a towelette elevating system for automatically positioning a stack of towelettes within a predefined distance of a dispensing aperture of a dispensing container, and automatically counteracting the weight of a stack of towelettes. A similar device is described in TW443981.

In yet another embodiment, STD barriers are separated by nonstick materials such as wax paper. Yet another embodiment, individual sheets are wrapped in a tearable package. This embodiment is particularly suited to those sheets that are combined with antiviral lubricants and other moist coatings. Moist coatings can include KY Jelly, Astroglide, silicone lubricant, petroleum jelly, baby oil, Crisco, olive, coconut oil or other vegetable or nut oils, and combinations thereof. In the simplest embodiment, the barriers can be dispensed in a zip lock bag.

In other embodiments, one or more edges of a precut sheet is coated with a non-self-stick material that can be easily grasped by the user. Alternatively, a small protruding tab can be coated with a non-stick material, and the tabs can be staggered for easy access. In yet another embodiment, paper or plastic tabs can be added to the sheets, for example with glue or heat welding.

Shape can also vary, although the square or rectangular is preferred because it is the most cost effective shape and can be used in a wide variety of intimate activities. However, the shape can vary as desired and includes circular (which may fit well over a penis) or the dumbbell or hourglass shape (which conforms to a woman's genital area) and allows good contact on the top and bottom edges against the users skin. The device can also include a small amount of adhesive along one or more edges, but this will add to the cost. Instead, it is preferred that the user apply a little lubricant to the skin, to which the STD barrier will adhere.

It is also possible to either sterilize the devices, e.g., with gamma radiation, or UV light, by treatment with a sterilizing chemical such as ozone or alcohol, or to make the devices in a sterile way. Films should be tested both before and after sterilization to confirm that the sterilization procedures do not negatively affect film performance characteristics.

In yet another embodiment, one side of the STD barrier is coated with various antimicrobial and/or antiviral materials, and the device can optionally be printed or colored on one side so as to indicate which side is applied to the genitals. Alternatively, the STD barrier can be bi- or multilayered, with one layer optimized for taste and the other for anti-infective properties.

In use the film is placed over the vulval area and care is taken not to reverse the film once used. As always, hands and nails and genitalia should be well cleaned before initiating any intimate contact, and care should be taken to avoid moving body parts, devices or the barrier's used side from one partner to the other. If desired, the device can be held in place with a small amount of compatible lubricant applied to the skin. Once used, the device is carefully disposed of.

### Example 1

### Film Selection

There are many commercially available films that can be tested for use in the invention, and the following tables list just a few:

| | | TRANSPORT | | | | VACUFLEX | |
|---|---|---|---|---|---|---|---|
| Property | Procedure | TX1540 | TX2060 | TX4100 | TX1400 | 184/0030 | 18411 |
| Film type | | polyurethane | copolyester | ether-amide | polyurethane | polyurethane | polyurethane |
| Specify | ASTM D- | 1.20 | 1.20 | 1.10 | 1.20 | 1.12 | 1.12 |
| gravity | 792 | | | | | | |
| Shore | ASTM D- | 80 A | 45 D | 40 D | 80 A | 80 A | 80 A |
| hardness | 2240 | | | | | | |
| Tensile | ASTM D- | 4000-5000 | 2000-3000 | 4500-5500 | 4000-5000 | 3800-5000 | 4200-5200 |
| strength, psi | 412 | | | | | | |
| Elongation, % | ASTM D- | 600-700 | 350-500 | 600-700 | 600-700 | 400-500 | 400-500 |
| | 412 | | | | | | |
| Modulus | ASTM D- | 550-650 | 650-750 | 650-750 | 500-650 | 800-1200 | 900-1100 |
| (100%), psi | 412 | | | | | | |
| MVT, | ASTM E-96 | — | 850 | — | — | N/A | N/A |
| g/m²/day | Procedure B | 900 | — | 875 | | | |
| 0.5 mil | (upright) | 800 | 725 | 775 | — | | |
| 0.6 mil | | | | | 725 | | |
| 1.0 mil | | | | | | | |

| Product | Product | Hardness | | | |
|---|---|---|---|---|---|
| Series | Type | (Shore A) | Product Features | Market(s) | Applications |
| A3600 | Aliphatic | 92 | High clarity film product for surface | Inflatables, | Road sign |
| | Ester | | protection | Laminates | coatings, |
| | | | | | Automotive |
| | | | | | stoneguard |
| A4700 | Aliphatic | 78 | Optical clarity, excellent bonding to | Glass | Security |
| | Ether | | polycarbonate & glass, has internal | Glazing | Glazing |
| | | | primer | | |
| PS7000 | Aromatic | 85 | Clear, excellent physical property matrix, | Belting, | Food Belting |
| | Ester | | good petrochemical resistance, | Inflatables | |
| | | | formulations available that comply with | Medical | |
| | | | FDA 177.2600 | | |
| PS8000 | Aromatic | 93-95 | Excellent “multi purpose” film product. | Belting, | Food Belting, |
| | Ester | | Excellent resistance to petrochemicals, | Inflatables | Emblems, |
| | | | good air retention properties, | Adhesives, | Labels, Tapes, |
| | | | formulations available that comply with | Medical | Medical |
| | | | FDA 177.2600 | | Inflatables, |
| | | | | | Liners, tarps |
| PS8400 | Aromatic | 85 | Good property matrix, good resistance to | Belting | Food Belting |
| | Ester | | petrochemicals. Formulation available | | |
| | | | that comply with 177.2600 | | |
| PS3100 | Aromatic | 78 | Soft, low melt, good adhesive | Adhesives, | Textile |
| | Ester | | characteristics | Inflatables | Adhesives, |
| | | | | | Adhesive |
| | | | | | Films, |
| | | | | | Emblems, |
| | | | | | Bladders |
| PT6100 | Aromatic | 83 | Excellent film product for laminates | Laminates | Medical barrier |
| | Ether | | using lighter light gauge film | | laminates |
| PT6200 | Aromatic | 90 | Excellent film product for laminates | Laminates | Barrier |
| | Ether | 90 | using lighter light gauge film, higher | | laminates |
| | | | melting range | | |
| PT7500 | Aromatic | 90 | Harder general purpose film product | Inflatables | Bladders, |
| | Ether | | | | Inflatables |
| PT9300 | Aromatic | 82 | Softer general purpose film product. | Laminates, | Barrier |
| | Ether | | Good light and heavy gauge performance | Medical, | Laminates, |
| | | | | Inflatables | Bladders, |
| | | | | | Textile |
| | | | | | adhesive |
| PT9200 | Aromatic | 85 | General purpose film product | Inflatables, | Bladders, Tent |
| | Ether | | | Thermoformed | Windows, |
| | | | | Covers | Tarps, Bags, |
| | | | | | Protective |
| | | | | | Covers |
| PT1710S | Aromatic | 80 | Soft hand monolithic TPU breathable | Breathables | Breathable |
| | Ether | | film for light gauge laminates | | barrier |
| | | | | | laminates |
| X1632 | Aromatic | 88 | Tougher monolithic TPU breathable film | Breathables | Breathable |
| | Ether | | for light gauge laminates | | barrier |
| | | | | | laminates |
| PS2000 | Copolyester | 75 | Soft hand monolithic Copolyester | Breathables | Breathable |
| | | | breathable film for light gauge laminates | | barrier |
| | | | | | laminates |
| PT5100 | Aromatic | 86 | High clarity general purpose film | Belting, | Tent windows, |
| | Ether | | product. Formulation available that | Inflatables | Bags |
| | | | comply with FDA 177.2600 | | |
| PT5510 | Aromatic | 87 | Inorganic non halogen opaque flame | Flame | Laminates, |
| | Ether | | retardant film product | Retardant | Bladders |
| PT9101 | Aromatic | 87 | Halogenated opaque flame retardant film | Flame | Laminates, |
| | Ether | | product | Retardant | Bladders |
| PT9700 | Aromatic | 85 | Organic non halogen clear flame | Flame | Laminates, |
| | Ether | | retardant film product | Retardant | Bladders |
| PS8601 | Aromatic | 95 | Halogenated opaque flame retardant film | Flame | Laminates, |
| | Ester | | product | Retardant | Bladders |

One thermoplastic polyurethane film was selected for proof of concept testing herein. It has the following characteristics, as determined by the supplier:

| | FILM |
|---|---|
| Type of film | Polyether polyurethane |
| Thickness | 1 mil (1 mil = 25.4 micron) |
| Manufacturer (product code) | American Polyfilm, Inc. MT 1001 |
| | SO#2845 |
| Durometer (Shore A scale) | 82 |
| Tensile Strength @ Break | 5,080 psi |
| Elast¹ | |
| Elongation @ Break Elast | 660% |
| Tear Strength Die C (lb/in) | 430 lb/in |
| Taste | none |
| Tactile Responsiveness | Good resilience, flexibility |

¹break elast refers to the elongation at which rupture occurs in the application of continued tensile stress (aka ultimate elongation).

### Example 2

### Viral Exclusion

Using test ASTM F1671 Section 5.4, latex, vinyl, neoprene and nitrile surgical gloves available from ANSELL™ have previously been shown to exclude virus. However these films have not yet been rigorously tested under the environmental conditions described herein.

Since viruses are generally smaller than microbes, it should suffice to test a small virus in exclusion studies. The virus selected was porcine parvovirus (PPV). PPV is a very small virus (18-24 nanometers), smaller than the herpes simplex virus (approx. 200 nm) and smaller than HIV (approx. 100 nm.) Viral exclusion was measured at body temperature without load (no elongation).

More particularly the film was tested for viral exclusion as follows:
4 mL of Hank's Buffered Salt Solution (HBSS) was added to each of 5 conical centrifuge tubes.
1 mL of porcine parvovirus (PPV) solution was placed into each of four test tubes for Positive Control and Test Samples.
1 mL of HBSS was placed into one test tube for Negative control.
Test tubes were covered with plastic film, which was secured with rubber bands.
Each test tube was inverted into a conical centrifuge tube containing HBSS.
Tubes were gently mixed on a platform rocker at 37° C., and the HBSS was assayed for infectious virus at 0, 30, 45 and 60 minutes.

The results are presented in the following table, but in short, PPV did not penetrate the plastic film barrier as indicated by the absence of infectious PPV in the HBSS of the 30, 45 and 60 minute test samples:

| | | Log₁₀ TCID₅₀/ | Log₁₀ TCID₅₀ |
|---|---|---|---|
| Sample | Volume (mL) | mL PPV | PPV¹ |
| Positive Control | 4.0 | 4.9 | 5.5 |
| Negative Control | 4.0 | ≦0.6 | ≦1.2 |
| 30 Minute Sample | 4.0 | ≦0.6² | ≦1.2 |
| 45 Minute Sample | 4.0 | ≦0.6 | ≦1.2 |
| 60 Minute Sample | 4.0 | ≦0.6 | ≦1.2 |

¹Total log10 TCID50 = log [10\hat{ }(log10 TCID50/mL) × mL]. For example, Total log10 TCID50 for the positive control was calculated as log(10\hat{ }4.9 × 4) = 4.9 log10 TCID50.
²All values followed by “≦” indicate that no virus was detected. The value represents the limit of detection of the assay.

The results described herein are preliminary and additional rigorous testing according to the standards described herein, including tests at 150% and greater elongation, higher temperatures, as well as barrier stability tests and tests with common lubricants, will be needed before such STD devices can be approved or cleared by the FDA for its intended use. However, these proof of concept tests indicate the feasibility of finding and/or developing films appropriate for use as an effective and pleasing STD barrier.

Each of the following references is hereby incorporated by reference in its entirety:

U.S. Pat. No. 4,949,731
U.S. Pat. No. 5,409,016
U.S. Pat. No. 5,655,543
U.S. Pat. No. 5,513,653

### JP8141008

U.S. Pat. No. 6,418,931

### GR1002601

U.S. Pat. No. 5,649,549
U.S. Pat. No. 4,735,621

### CN1230392

### JP7178129

U.S. Pat. No. 5,534,346
U.S. Pat. No. 5,358,935, U.S. Pat. No. 5,948,432, U.S. Pat. No. 6,124,265, U.S. Pat. No. 6,914,126, U.S. Pat. No. 6,989,437
U.S. Pat. No. 6,053,357

### WO0065972

### US2004169047

U.S. Pat. No. D452,788S
U.S. Pat. No. 5,768,968
U.S. Pat. No. 4,340,162
U.S. Pat. No. 7,216,775

### WO2004052164

### JP2007244588

U.S. Pat. No. 5,664,677

### US2006060599

TW443981

What is claimed is:

1. A sexually transmitted disease (Std) barrier, comprising a planar sheet of biocompatible material having essentially no taste, a durometer of 70-85, a tensile strength of at least 3000 psi, a reversible elongation stretch of at least 150%, that excludes virus and microbes at 37° C.

2. The STD barrier of claim 1, wherein the planar sheet comprises a thermoplastic polyurethane film.

3. The STD barrier of claim 1, wherein the planar sheet comprises polyether polyurethane.

4. The STD barrier of claim 1, wherein the planar sheet comprises 1 mil polyether polyurethane having a Durometer of 82, a tensile strength of at least 5000 psi, a reversible elongation stretch of at least 150%, a tear strength of at least 400 lb/inch, and no taste.

5. The STD barrier of claim 1, wherein the planar sheet comprises a keratin film.

6. The STD barrier of claim 1, wherein the planar sheet comprises a keratin-silicone film.

7. The STD barrier of claim 1, that is precut and interleaved for dispensing.

8. The STD barrier of claim 1, that is formed in a continuous roll that is perforated for dispensing.

9. The STD barrier of claim 1, that about 12 inches by 12 inches when dispensed.

10. The STD barrier of claim 1, that is individually wrapped.

11. The STD barrier of claim 1, that is dumbbell shaped.

12. The STD barrier of claim 1, that has adhesive along one or more edges.

13. The STD barrier of claim 1, that is about 200-200 um in thickness.

14. The STD barrier of claim 1, that is coated on one side with an anti-infective compound.

15. The STD barrier of claim 1, that is coated on one side with a flavorant.

16. The STD barrier of claim 1, that excludes virus and microbes when tested at 150% elongation.

17. The STD barrier of claim 1, that excludes virus and microbes when tested at 200% elongation.

18. The STD barrier of claim 1, that excludes virus and microbes when tested at 500% elongation.

19. The STD barrier of claim 1, that has a durometer of less than 80.

20. The STD barrier of claim 1, wherein said planar sheet comprises a first biocompatible and anti-infective film adjacent a second biocompatible film having no taste.

21. An STD barrier, comprising a planar sheet of biocompatible polyether polyurethane, said sheet having about 1 mil thickness, having essentially no taste, a durometer of about 82, a tensile strength of at least 5000 psi, a reversible elongation stretch of at least 600%, a tear strength of at least 400 lb/inch, and that excludes virus and microbes when tested at 37° C., and that is square or rectangular shape measuring about 12 inches on a side.

22. An STD barrier, comprising a planar sheet of biocompatible keratin based film, said sheet having essentially no taste, a durometer of about 75-85, a tensile strength of at least 3000 psi, a reversible elongation stretch of at least 100%, and that excludes virus and microbes when tested at 37° C., and that is square or rectangular shape measuring about 12 inches on a side.
